(19) United States
(12) Patent Application Publication
(10) Pub. No.: US 2022/0168300 A1
(43) Pub. Date: Jun. 2, 2022

(54) Methods of Managing Vascular Conditions and Diabetic Peripheral Neuropathies

(71) Applicant: Emory University, Atlanta, GA (US)

(72) Inventors: Young-sup Yoon, Atlanta, GA (US); Ji Woong Han, Decatur, GA (US); Hyunsuk Shim, Atlanta, GA (US)

(21) Appl. No.: 17/430,624

(22) PCT Filed: Feb. 12, 2020
(86) PCT No.: PCT/US2020/017833

§ 371 Date: Aug. 12, 2021

Related U.S. Application Data
(60) Provisional application No. 62/804,736, filed on Feb. 12, 2019.

(51) Int. Cl. A61K 31/506; A61K 31/18; A61K 31/167; A61K 35/12; A61P 3/10; A61P 25/02

(52) U.S. Cl. CPC A61K 31/506; A61K 31/18; A61P 25/02; A61K 35/12; A61P 3/10; A61K 31/167

(57) ABSTRACT

This disclosure relates to managing diabetic neuropathy using compounds disclosed herein. In certain embodiments, this disclosure relates to methods of treating or preventing vascular conditions such as diabetic neuropathy comprising administering a compound capable of epigenetic modification, such as histone deacetylase inhibitors, to a subject in need thereof. In certain embodiments, this disclosure relates to the use of HDAC inhibitors to treat directly diabetic neuropathies and pain without the use of progenitor cells or stem cells. In certain embodiments, the subject is at risk of, exhibiting symptoms of, or diagnoses with diabetes, diabetic neuropathy, peripheral neuropathy, autonomic neuropathy, radiculoplexus neuropathy, mononeuropathy, diabetic retinopathy, or complications related thereto. In certain embodiments, the compound is selected from belinostat, quisinostat, and vorinostat.

### CROSS-REFERENCE TO RELATED APPLICATIONS

This application claims the benefit of U.S. Provisional Application No. 62/804,736 filed Feb. 12, 2019. The entirety of this application is hereby incorporated by reference for all purposes.

### STATEMENT REGARDING FEDERALLY SPONSORED RESEARCH OR DEVELOPMENT

This invention was made with government support under DK094346 awarded by the National Institutes of Health. The government has certain rights in the invention.

### BACKGROUND

Many cardiovascular disease patients have diabetes as a co-morbid disease. Diabetic neuropathy is nerve damage that results from having high blood sugar. Glucose can injure nerves throughout your body; however, often it damages nerves in legs and feet first. Symptoms of diabetic neuropathy can range from pain and numbness to problems with digestion, urinating, and the vascular system. Certain drugs can ease mild to moderate pain caused by diabetic nerve damage. However, some may raise your risk of heart attack, stroke, or kidney damage. Thus, there is a need to identify improved therapies.

Prattichizzo et al. report an epigenetic mechanism of endothelial progenitor cell dysfunction in type 2 diabetes. J Clin Epigenet, 2015, 7(1): 56.

Han et al. report cell therapy for diabetic neuropathy using adult stem or progenitor cells. Diabetes Metab J., 2013, 37(2): 91-105. Han et al. also report bone marrow-derived mesenchymal stem cells improve diabetic neuropathy by direct modulation of both angiogenesis and myelination in peripheral nerves. See Cell Transplant, 2016, 25(2):313-26. See also U.S. Published Patent Application Nos. 2015/0259649, 2017/0157178 and U.S. Pat. No. 9,458,131.

Tan et al. report histone deacetylase inhibitors promote endothelial nitric oxide synthase expression in vascular smooth muscle cells. J Cell Mol Med. 2017, 21(9):2022-2035.

References cited herein are not an admission of prior art.

### SUMMARY

This disclosure relates to managing diabetic neuropathy using compounds disclosed herein. In certain embodiments, this disclosure relates to methods of treating or preventing vascular conditions and diabetic neuropathy comprising administering a compound capable of epigenetic modification, such as histone deacetylase inhibitors, to a subject in need thereof. In certain embodiments, this disclosure relates to the use of HDAC inhibitors to treat directly diabetic neuropathies and pain without the use of progenitor cells or stem cells. In certain embodiments, the subject is at risk of, exhibiting symptoms of, or diagnoses with diabetes, diabetic neuropathy, peripheral neuropathy, autonomic neuropathy, radiculoplexus neuropathy, mononeuropathy, diabetic retinopathy, or complications related thereto. In certain embodiments, the compound is selected from belinostat, quisinostat, and vorinostat.

In certain embodiments, the subject is at risk of, exhibiting symptoms of, or diagnosed with diabetic neuropathy, diabetes, or prediabetes. In certain embodiments, the agent is administered in the absence of autologous stem cells or the agent is administered in combination with autologous stem cells.

In certain embodiments, this disclosure relates to methods of treating or preventing diabetic nerve pain comprising administering an effective amount of an epigenetic modifying agent to a subject in need thereof. In certain embodiments, the epigenetic modifying agent is a histone deacetylase inhibitor. In certain embodiments, the subject is at risk of, exhibiting symptoms of, or diagnoses with diabetic nerve pain. In certain embodiments, the compound is selected from belinostat, quisinostat, and vorinostat.

In certain embodiments, this disclosure relates to methods of reducing pain comprising administering an effective amount of an epigenetic modifying agent to a subject in need thereof. In certain embodiments, the epigenetic modifying agent is a histone deacetylase inhibitor. In certain embodiments, the agent is belinostat. In certain embodiments, the agent is vorinostat. In certain embodiments, the agent is quisinostat. In certain embodiments, the subject is at risk of, exhibiting symptoms of, or diagnosed with diabetic neuropathy, diabetes, or prediabetes. In certain embodiments, the agent is administered in the absence of autologous stem cells or the agent is administered in combination with autologous stem cells.

### BRIEF DESCRIPTION OF THE DRAWINGS

FIG. 1A shows data indicating direct injection of the HDAC inhibitor (HDACi) belinostat improved nerve conduction velocities (NCVs). Six weeks after streptozotocin injection, sensory NCVs in diabetic mice were significantly lower than in age-matched nondiabetic mice.

FIG. 1B shows data indicating diabetic mice showed decreased motor NCVs from 11 weeks after diabetes induction. Direct injection of belinostat significantly improved motor NCVs in diabetic mice over 15 weeks.

FIG. 2A shows data indicating direct injection of belinostat improved tactile allodynia and prevented thermal hypoalgesia in diabetic nerves. At 14 weeks after belinostat injection, paw withdrawal thresholds were recovered to nondiabetic group.

FIG. 2B shows data indicating thermal withdrawal latency was also maintained in HDAC treated group.

FIG. 2C shows data of a rotarod performance study showed no significant difference between DM and DM+HDACi group, while diabetic groups (DM and DM-HDACi) showed decreased fall latency compared to nondiabetic group (NDM).

FIG. 3 shows data on the density of intraepidermal nerve fiber (IENF) in hind paw plantar skin indicating an effect of belinostat on loss of intraepidermal nerve fibers induced by diabetes. Images were taken of PGP9.5 staining of IENF and DAPI in the plantar skin of the hind paw. Nondiabetic mouse injected with DMSO (NDM). Diabetic mouse injected with DMSO (DM). Diabetic mouse injected with belinostat (DM+HDACi).

FIG. 4A shows data indicating the degenerative structure of myelin in diabetic nerves restored by belinostat injection. Toluidine blue-stained cross sections of sciatic nerves of nondiabetic mouse injected with DMSO, diabetic mouse injected with DMSO, and diabetic mouse injected with belinostat. Electron microscopy of sciatic nerve cross-sections from nondiabetic mouse injected with DMSO, diabetic mouse injected with DMSO, and diabetic mouse injected with belinostat. The ratio of abnormal fiber in sciatic nerve in each group were determined. (top: decompaction, top/middle: infolding, middle/bottom: focal lysis, bottom: splitting).

FIG. 4B shows data on the circularity of axon in sciatic nerve.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Prior to describing the various embodiments, the following definitions are provided and should be used unless otherwise indicated.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In preferred embodiment, the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

As used herein, “subject” refers to any animal, preferably a human patient, livestock, or domestic pet.

As used herein, the terms “prevent” and “preventing” include the prevention of the recurrence, spread or onset. It is not intended that the present disclosure be limited to complete prevention. In some embodiments, the onset is delayed, or the severity is reduced.

As used herein, the terms “treat” and “treating” are not limited to the case where the subject (e.g. patient) is cured and the disease is eradicated. Rather, embodiments of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

The term “effective amount” or “therapeutically effective amount” refers to that amount of a compound or pharmaceutical composition described herein that is sufficient to effect the intended application including, but not limited to, disease treatment. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on, for example, the particular compounds chosen, the dosing regimen to be followed, whether it is administered in combination with other agents, timing of administration, the tissue to which it is administered, and the physical delivery system in which it is carried.

### Methods of Use

This disclosure relates to managing diabetic neuropathy using compounds disclosed herein. In certain embodiments, this disclosure relates to methods of treating or preventing diabetic neuropathy comprising administering a compound capable of epigenetic modification, such as histone deacetylase inhibitors, to a subject in need thereof. In certain embodiments, the subject is at risk of, exhibiting symptoms of, or diagnoses with diabetes, diabetic neuropathy, peripheral neuropathy, autonomic neuropathy, radiculoplexus neuropathy, mononeuropathy, diabetic retinopathy, or complications related thereto. In certain embodiments, the compound is selected from belinostat, vorinostat, and quisinostat.

In certain embodiments, this disclosure relates to methods of treating or preventing peripheral vascular disease, myocardial ischemia, heart failure, diabetic wounds, diabetic and stroke comprising administering an effective amount of an epigenetic modifying agent to a subject in need thereof. In certain embodiments, the epigenetic modifying agent is a histone deacetylase inhibitor. In certain embodiments, the subject is at risk of, exhibiting symptoms of, or diagnoses with vascular disease, myocardial ischemia, heart failure, diabetic wounds, diabetic and stroke. In certain embodiments, the compound is selected from belinostat, quisinostat, and vorinostat.

In certain embodiments, this disclosure relates to methods of treating or preventing peripheral vascular disease associated with eyes (retinopathy), nerves (neuropathy), or kidneys (nephropathy) comprising administering an effective amount of an epigenetic modifying agent to a subject in need thereof. In certain embodiments, the epigenetic modifying agent is a histone deacetylase inhibitor. In certain embodiments, the subject is at risk of, exhibiting symptoms of, or diagnoses with retinopathy, neuropathy, or nephropathy. In certain embodiments, the compound is selected from belinostat, quisinostat, and vorinostat.

In certain embodiments, this disclosure relates to methods of treating or preventing peripheral vascular disease associated with hypertension (high blood pressure), diabetic nephropathy (kidney damage due to diabetes), and congestive heart failure comprising administering an effective amount of an epigenetic modifying agent to a subject in need thereof. In certain embodiments, the epigenetic modifying agent is a histone deacetylase inhibitor. In certain embodiments, the subject is at risk of, exhibiting symptoms of, or diagnoses with hypertension, diabetic nephropathy, and congestive heart failure. In certain embodiments, the compound is selected from belinostat, quisinostat, and vorinostat.

In certain embodiments, the compound capable of epigenetic modification is a DNA methyltransferase inhibitor, a histone deacetylase inhibitor (HDACi), DNA methylation inhibitor, a Rho-associated kinase (ROCK) inhibitor, Wnt inhibitor, GSK-3beta inhibitor, and/or a dihydropyridine. In certain embodiments, the DNA methyltransferase inhibitor is N-phthalyl-L-tryptophan (RG 108). In certain embodiments, the DNA methylation inhibitor is 5-azacitidine or decitabine.

In certain embodiments, the HDAC inhibitor is belinostat-(E)-N-hydroxy-3-[3-(phenylsulfamoyl)phenyl]prop-2-enamide, quisinostat-N-hydroxy-2-(4-((((1-methyl-1H-indol-3-yl)methyl)amino)methyl)piperidin-1-yl)pyrimidine-5-carboxamide, vorinostat-suberoylanilide hydroxamic acid (SAHA), entinostat-pyridin-3-ylmethylN-[[4-[(2-aminophenyl)carbamoyl]phenyl]methyl]carbamate, panobinostat-(E)-N-hydroxy-3-[4-[[2-(2-methyl-1H-indol-3-yl)ethylamino]methyl]phenyl]prop-2-enamide, mocetinostat-N-(2-aminophenyl)-4-[[(4-pyridin-3-ylpyrimidin-2-yl)amino]methyl]benzamide, Romidepsin, 7-ethylidene-4,21-di(propan-2-yl)-2-oxa-12,13-dithia-5,8,20,23-tetrazabicyclo[8.7.6]tricos-16-ene-3,6,9,19,22-pentone, givinostat-(6-((diethylamino)methyl)naphthalen-2-yl)methyl(4-(hydroxycarbamoyl)phenyl)carbamate, trichostatinA(TSA)-[R-(E,E)]-7-[4-(Dimethylamino)phenyl]-N-hydroxy-4,6-dimethyl-7-oxo-2,4-heptadienamide, valproic acid (VPA) or salts of any compound disclosed herein.

In certain embodiments, the ROCK inhibitor is 4-(1-aminoethyl)-N-(pyridin-4-yl)cyclohexanecarboxamide (Y-27632) or salt thereof. In certain embodiments, the dihydropyridine is 1,4-dihydro-2,6-dimethyl-5-nitro-4-[2-(trifluoromethyl)phenyl]-3-pyridinecarboxylic acid, methyl ester (BayK8644), ester, derivative, or a salt thereof. In certain embodiments, the GSK-3beta inhibitor is 6-[[2-[[4-(2,4-dichlorophenyl)-5-(5-methyl-1H-imidazol-2-yl)-2pyrimidinyl]amino]ethyl]amino]-3-pyridinecarbonitrile (CHIR99201) or salt thereof.

Diabetic neuropathy can be categorized into subtypes related to which nerves are affected. Peripheral neuropathy affects the feet and legs first, followed by the hands and arms. Signs and symptoms of peripheral neuropathy are often worse at night, and may include: numbness or reduced ability to feel pain or temperature changes, tingling or burning sensation, sharp pains or cramps, increased sensitivity to touch, muscle weakness, loss of reflexes, e.g., in the ankle, loss of balance and coordination, ulcers, infections, and bone and joint pain in the feet.

Autonomic neuropathy affects the heart, bladder, stomach, intestines, sex organs and eye possibly causing bladder problems, including urinary tract infections or urinary retention or incontinence, constipation, uncontrolled diarrhea, slow stomach emptying (gastroparesis), causing nausea, vomiting, bloating and loss of appetite, difficulty swallowing, increased or decreased sweating, problems controlling body temperature, changes in the way your eyes adjust from light to dark, increased heart rate at rest, sharp drops in blood pressure after sitting or standing that may cause you to faint or feel lightheaded, erectile dysfunction, vaginal dryness, decreased sexual response, or combinations thereof.

Radiculoplexus neuropathy, otherwise known as diabetic amyotrophy, femoral neuropathy or proximal neuropathy, affects nerves in the thighs, hips, buttocks or legs. Subjects may have type 2 diabetes and/or over 60 years old. Symptoms may be one side of the body or may spread to the other side. Symptoms include severe pain in a hip and thigh or buttock, weak and shrinking thigh muscles, difficulty rising from a sitting position, abdominal swelling and weight loss. Pain may occur for more than 24 hours.

Mononeuropathy, otherwise known as focal neuropathy, affects a nerve in the face, middle of the body (torso) or leg. Mononeuropathy may cause severe pain. Symptoms include pain in the shin or foot, lower back or pelvis, front of thigh, chest or abdomen. Mononeuropathy may also cause nerve problems in the eyes and face, leading to difficulty focusing, double vision, aching behind one eye, paralysis on one side of your face (Bell's palsy), and carpal tunnel (numbness or tingling in your hand or fingers, except your pinkie.

Diabetes may cause develop neuropathy, risk factors are associated with nerve damage include poor blood sugar control, kidney disease, having a body mass index (BMI) greater than 24, smoking, or combinations thereof.

Diabetic neuropathy can cause a number of serious complications, including loss of a toe, foot or leg, joint damage, urinary tract infections and urinary incontinence, hypoglycemia unawareness, sharp drops in blood pressure, digestive problems, sexual dysfunction, and increased or decreased sweating.

As used herein, the term “diabetes” refers to diabetes mellitus, which can be type 1 or type 2 diabetes, or gestational diabetes. Type 1 refers to a subject that fails to produce sufficient insulin. Type 2 refers to subjects that become resistant to insulin. Diabetes mellitus results in persistent hyperglycemia that produces reversible and irreversible pathologic changes within the microvasculature of various organs.

Diabetics often develop visual dysfunctions such as diabetic retinopathy, glaucoma, cataracts, macular edema, abnormal color vision, and decreased contrast sensitivity. Diabetic retinopathy is traditionally characterized as a retinal microvascular disease that is manifested as a cascade of stages with increasing levels of severity and worsening prognoses for vision. Major risk factors reported for developing diabetic retinopathy include the duration of diabetes mellitus, quality of glycemic control, and presence of systemic hypertension.

In certain embodiments, a subject may be in need thereof because the subject has recurrent abnormal blood sugar levels, diabetes, prediabetes, or recurrent abnormally high blood sugar levels. A normal fasting (no food for eight hours) blood sugar level is between 70 and 99 mg/dL. A normal blood sugar level two hours after eating is less than 140 mg/dL. Recurrent abnormal levels may be for more than a month, or more than three months, or more than six months, or more than a year.

In certain embodiments, the subject is diagnosed with diabetes or pre-diabetes. Diabetes is typically diagnosed by an indication of abnormally high blood sugar levels. Some examples include: two consecutive fasting blood glucose tests that are equal to or greater than 126 mg/dL; any random blood glucose that is greater than 200 mg/dL; A1c test, i.e., measure of a percentage of the glycated hemoglobin, that is equal to or greater than 6.5 percent; or a two-hour oral glucose tolerance test with any value over 200 mg/dL. Pre-diabetes is typically diagnosed by a higher than normal blood sugar level below the amounts indicated above. Some examples include: a fasting blood glucose in between 100-125 mg/dL; an A1c between 5.7-6.4 percent; and between 140 mg/dL and 199 mg/dL during a two-hour 75 g oral glucose tolerance test.

In certain embodiments agent disclosed herein are administered by the mouth (orally) or delivered directly into the stomach (gastric gavage), delivered into a blood vessel (intravenous); delivered onto, into, under, or across the skin or into a muscle (epicutaneous, intradermal, subcutaneous, transdermal, and intramuscular administration, respectively); instilled onto or into the eye (transcorneal or intraocular, respectively); into the brain (intracerebral) or the space surrounding the dura mater or that surrounding the distal spinal cord (epidural and intrathecal, respectively); administered into the peritoneal cavity (intraperitoneal), directly into the marrow cavity (intraosseous); sprayed into the nose for absorption across the nasal mucous membranes or into the lungs (intranasal) or delivered into the lungs by direct tracheal instillation (intratracheal) or inhalation; or using other body orifices or surgical exposures.

In certain embodiments, the disclosure relates to methods of generating epigenetically altered cells comprising mixing isolated cells with compositions disclosed herein under conditions such that epigenetically altered cells are formed. In certain embodiments, methods of treating or preventing vascular or diabetic diseases or conditions are contemplated.

In certain embodiments, the disclosure contemplates methods of epigenetically modifying stem or progenitor cells comprising mixing the stem or progenitor cells and compositions comprising compounds disclosed wherein such as a 5-aza-2′-deoxycytidine, or/and N-phthalyl-L-tryptophan (RG108), under conditions such that cells with enhanced angiogenic gene expression are produced. In certain embodiments, the conditions are such that reduced DNA methylation in the promoters of angiogenic genes occurs. In certain embodiments, the angiogenic genes are one or more or all of the genes selected from Akt1, Hgf, Mapk14, Sphk1, Vegfc, Nudt6, Kdr, Vegfa, and Pten.

In certain embodiments, the stem or progenitor cells are mesenchymal stem cells (MSCs) or endothelial progenitor cells (EPCs), cardiac stem cells, myoblasts, adult bone marrow-derived cells, umbilical cord blood cells, fibroblasts, or peripheral blood CD34+ cells. In certain embodiments, the endothelial progenitor cells are bone marrow derived. In certain embodiments, the cells are obtained from a subject diagnosed with diabetes and/or cardiovascular disease.

In certain embodiments, the disclosure contemplates methods of treating or preventing vascular disease or condition comprising: mixing progenitor cells from a subject and a histone deacetylase (HDAC) inhibitor as provided herein under conditions such that epigenetically modified cells with enhanced angiogenic gene expression are produced; and administering an effective amount of a composition comprising the epigenetically modified cells or cells cultured therefrom to a subject in need thereof. In certain embodiments, the progenitor cells are bone marrow derived cells, endothelial progenitor cells, or mesenchymal stem cells. In certain embodiments, the progenitor cells were obtained from the subject receiving the administered composition. In certain embodiments, the vascular disease or condition is peripheral vascular disease, myocardial ischemia, cardiovascular disease, heart failure, or stroke.

In certain embodiments, the disclosure contemplates methods of treating or preventing a diabetic disease or conditions comprising: mixing progenitor cells from a subject and a histone deacetylase inhibitor as provided herein under conditions such that epigenetically modified cells with enhanced angiogenic gene expression are produced; and administering an effective amount of a composition comprising the epigenetically modified cells or cells cultured therefrom to a subject in need thereof. In certain embodiments, the progenitor cells are bone marrow derived cells, endothelial progenitor cells, or mesenchymal stem cells. In certain embodiments, the progenitor cells were obtained from the subject receiving the administered composition. In certain embodiments, the diabetic disease or condition is diabetic wounds or diabetic neuropathy.

In certain embodiments, the epigenetically modified cells may be cultured, expanded, or replicated in order to provide enhanced concentrations upon administration/transplantation and the modified cells may be autologous (i.e., derived from the person on whom they are used) or allogeneic (i.e., originating from another person) in origin. In certain embodiments, methods include those subjects that are co-morbid with a vascular disease or condition and a diabetic disease or condition.

In certain embodiments, the disclosure contemplates intravenous injection and direct infusion into the coronary arteries. In certain embodiments, the methods can be used in subject whose blood flow has been restored to their hearts after a heart attack. In certain embodiments, the compositions are injected directly into the ventricular wall of the subject, i.e., endo-myocardial injection or into the peritoneal cavity, and may be carried out either via a catheter or during open-heart surgery.

In certain embodiments, the disclosure contemplates methods of treating or preventing vascular disease or condition comprising: mixing progenitor cells from a subject and a histone deacetylase inhibitor as provided herein under conditions such that epigenetically modified cells with enhanced angiogenic gene expression are produced; and administering an effective amount of a composition comprising the epigenetically modified cells or cells cultured therefrom to a subject in need thereof in combination with administering effective amounts of pharmaceutical compositions disclosed herein comprising a histone deacetylase inhibitor or mixture of compounds to a subject in need thereof.

In certain embodiments, the disclosure contemplates methods of treating or preventing a diabetic disease or conditions comprising: mixing progenitor cells from a subject and a histone deacetylase inhibitor as provided herein under conditions such that epigenetically modified cells with enhanced angiogenic gene expression are produced; and administering an effective amount of a composition comprising the epigenetically modified cells or cells cultured therefrom to a subject in need thereof in combination with administering effective amounts of pharmaceutical compositions disclosed herein comprising a histone deacetylase (HDAC) inhibitor or mixture of compounds to a subject in need thereof.

In certain embodiments, the progenitor cells are bone marrow derived cells, endothelial progenitor cells, or mesenchymal stem cells. In certain embodiments, the progenitor cells were obtained from the subject receiving the administered composition. In certain embodiments, the diabetic disease or condition is diabetic wounds or diabetic neuropathy.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

### Examples

Rescue of epigenetic silencing of angiogenic genes in D-EPCs Diabetic EPCs (D-EPCs) have high methylation in the promoter regions of key angiogenic and neurotrophic genes and small molecular epigenetic regulators can reverse these changes, increase gene expression and improve cellular functions. To prepare D-EPCs streptozotocin (STZ) was used to induce diabetic conditions. Differential expression of angiogenic genes was investigated between normal EPCs (N-EPCs) and D-EPCs using an Angiogenesis Microarray kit. The expression of many angiogenic genes was suppressed in D-EPCs. In this disclosure, histone deacetylase (HDAC) inhibitors, i.e., Vorinostat (Zolinza, SAHA), Belinostat (Beleodaq, PXD-101), and Quisinostat (JNJ-26481585), were used. The preliminary experiments were performed using rat EPCs as well as human and rat MSCs. To test the reversible changes of gene expression and epigenetic modifications in D-EPCs by small molecule epigenetic regulators, epigenetic profiling studies were performed using methylation-sensitive restriction enzyme (MSRE)-PCR assays. An HDAC inhibitor, valproic acid (VPA), was used as the epigenetic regulator. D-EPCs showed high levels of DNA methylation in the promoters of angiogenic genes, consistent with the microarray results. VPA reduced DNA methylation at these promoters and enhanced gene expression.

Restoration of Impaired Gene Expression in Diabetic HUVECs, Schwann Cells, and DRG Neurons with HDAC Inhibitors.

Inspired by the powerful role of certain HDAC inhibitors on recovery of diabetic MSCs, experiments were performed using HUVECs, Schwann cells, and DRG neurons cultured under high glucose conditions to mimic diabetic environment. These three cell types were chosen because these are the target cells: endothelial cells (HUVEC), neuronal cells (DRG neuron, ND7/23), and glial cells (Schwann cell, S16), which are affected in diabetic neuropathy (DN). A variety of HDAC inhibitors were used (Vorinostat, Quisinostat, and Belinostat) to enhance the angiogenic, myelin-related, and neurotrophic gene expression, which was reduced in diabetic conditions. These compounds display pleiotropic activities, including the ability to restore defective gene expression involved in cell cycle control, apoptosis, growth signaling, invasion/metastasis, angiogenesis and immune recognition. Vorinostat, also known as suberoylanilide hydroxamic acid (SAHA), was identified based on its ability to induce differentiation of cultured murine erythroleukemia cells. Additional studies showed that SAHA inhibited tumor growth in rodents and could induce re-differentiation and growth arrest in breast cancer cells. SAHA inhibits HDAC 1-3, 6, and 8, selectively increasing expression of pro-apoptotic members of the Bcl-2 family and decreasing expression of genes involved in cell cycle progression such as CDK2, CDK4, cyclin D1, and cyclin D2 that also have anti-apoptotic activity. SAHA also increases expression of tumor necrosis factor and induces acetylation of the chaperone protein Hsp90, leading to cellular stress and apoptosis.

Importantly for brain tumor investigations, SAHA can cross the blood-brain-barrier (BBB), leading to increases in H3/H4 acetylation in brain tissue. SAHA was FDA approved in 2006 for treatment of cutaneous T-cell lymphoma. Responses have been seen in other hematologic malignancies including Hodgkin's lymphoma and myeloid malignancies. Ongoing trials are testing SAHA in breast, colon, and lung cancers. Another HDAC inhibitor, belinostat, is approved for the treatment of patients with relapsed or refractory peripheral T-cell lymphoma (PTCL).

To determine effects of different types of HDAC inhibitors on different target cells, cells were cultured in diabetic condition (450 mg/dl glucose, 7 days) and were treated HDAC inhibitors for 7 days. Quantitative RT-PCR demonstrated that mRNA expression levels of each cell type specific markers and functional genes were significantly decreased in diabetic condition and restored by HDAC inhibitor treatment. These data clearly suggest that these HDAC inhibitors can directly applied for treating diabetic neurovascular complications.

### Direct Injection of Belinostat Improves Nerve Conduction Velocities in Diabetic Neuropathy

To investigate the preventive therapeutic effects of direct injection of belinostat on diabetic nerves, streptozotocin (STZ)-induced diabetic mice were randomly assigned to solvent-treated (DM) as vehicle controls or HDAC inhibitor-treated (DM+HDACi) groups. Belinostat was selected as HDAC inhibitor for the animal diabetic neuropathy (DN) model as it had superior properties compared to vorinostat and quisinostat for upregulation of gene expression in vitro experimental data using cells. Belinostat was resuspended in 45% (2-hydroypropyl)-beta-cyclodextrin (HP-β-CD) and injected into mice via the intraperitoneal route at 50 mg/kg/day for 4 weeks. Treatment of HDAC inhibitor did not significantly alter blood glucose levels and animal body weight compere to DM group.

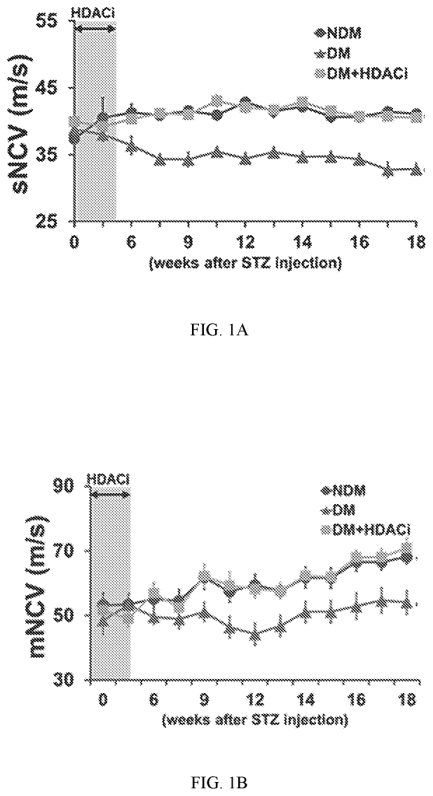

After intraperitoneal injection of belinostat or an equal volume of solvent vehicle (HP-β-CD), both sensory and motor nerve conduction velocities (NCVs) were measured for 18 weeks at every week (n=11-14, each group) from 0 week after treatment. Non-diabetic mice, which received solvent vehicle (HP-j-CD), were used as controls (NDM). Electrophysiological study showed about a 12% decrease in sensory nerve conduction velocities (NCVs) of DM group at 6 weeks of diabetes and a 20% decrease in motor NCVs of DM group at 11 weeks of diabetes, indicating development of a peripheral neuropathy (NDM versus DM: sensory NCV, 41.29±1.21 m/s versus 36.38±1.33 m/s; motor NCV, 57.40±2.51 m/s versus 46.36±3.92 m/s; P<0.05 for both). After treated with HDAC inhibitor, NCVs of DM+HDACi group maintained normal levels over 15 weeks (sensory NCV, 34.76±0.61 m/s; P<0.001 versus DM; motor NCV, 51.15±2.60 m/s; P<0.05 versus DM; both not significantly different from NDM) (FIGS. 1A and 1B). These data indicate that HDAC inhibitor could effectively prevent decrease of NCVs in diabetic mice.

### Direct Injection of Belinostat Ameliorates Tactile Allodynia and Prevents Thermal Hypoalgesia.

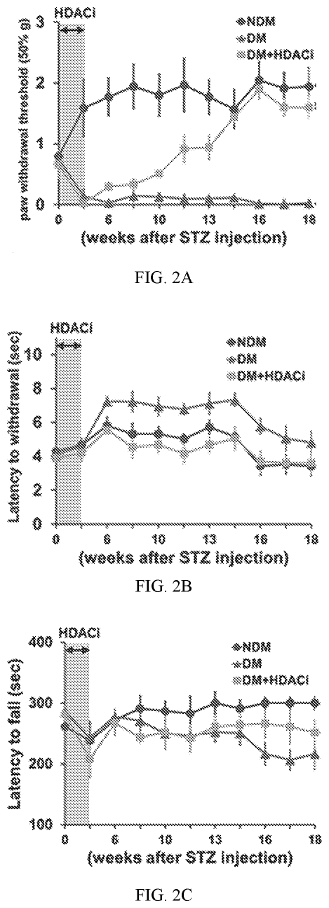

To investigate preventive effects of belinostat in diabetic neuropathy, tactile response threshold, thermal withdrawal latency, and rotarod performance were tested. For assay of tactile allodynia, mice were transferred to a glass cage with a wire mesh floor allowing access to the plantar surface of the hind paws. Behavioral accommodation was allowed for approximately 15 min until cage exploration and major grooming activities ceased. A monofilament is applied perpendicularly to the plantar surface of the hind paw until it buckles, delivering a constant pre-determined force (typically 0.2-13.7 mN) for 2-5 s. A response is considered positive if the animal exhibits any nocifensive behaviors, including brisk paw withdrawal, licking, or shaking of the paw, either during application of the stimulus or immediately after the filament is removed. Evaluation of response to von Frey hair testing demonstrated a significance decrease at 3 weeks of diabetes (NDM versus DM: 1.52±0.36 g versus 0.15±0.09 g; NDM versus DM+HDACi: 1.52±0.36 g versus 0.15±0.09 g; P<0.001 for both). At 14 weeks after HDAC inhibitor injection, DM+HDACi group recovered their paw withdrawal thresholds as NDM group (1.57±0.33 g versus 1.33±0.30 g; not significantly different), while DM group maintained similar decrease level of paw withdrawal thresholds at 14 weeks (NDM versus DM: 1.57±0.33 g versus 0.05±0.02 g; DM versus DM+HDACi: 0.05±0.02 g versus 1.33±0.30 g; P<0.001 for both) (FIG. 2A).

For measurement of paw thermal withdrawal latency, mice were placed in an observation chamber on top of the thermal testing apparatus and allowed to acclimate to the warmed glass surface (30° C.) and surroundings for 15 min. The mobile heat source was maneuvered to below the center of the right hind paw and turned on, a process that activates a timer and locally warms the glass surface at a rate of approximately 1° C./s. When the mouse withdrew the paw, movement sensors stopped the timer and turned off the heat source. Right hind paw was measured three times and the mean of three measurements used as a composite source for each mouse. The response latency was converted to the response temperature using a time: floor temperature calibration curve that was constructed each day to allow for day-to-day variations in the surface heating rate. Paw thermal withdrawal latency study showed an increase in withdrawal temperature of DM group at 12 weeks of diabetes (NDM versus DM: 34.19±0.34° C. versus 36.80±0.45° C.; DM versus DM+HDACi: 36.80±0.45° C. versus 34.30±0.24° C.; P<0.001 for both). Paw withdrawal temperature of DM+HDACi group was similar to that of nondiabetic control group (NDM) over 18 weeks, suggesting that the efficacy of HDAC inhibitor against thermal hypoalgesia (FIG. 2B).

For rotarod performance test, mice were placed on a rotarod apparatus to assess motor coordination and physical condition. Before the training session, each mouse was habituated to stay on the spindle with slow constant speed for 5 min. Mice were trained for 5 min at 45 rpm before measurement. The horizontal rod rotating by 45 rpm and the maximal length of observation time for each trial was 5 min. In rotarod performance study, there was no significant difference between DM and DM+HDACi group (256.61±21.99 sec versus 254.77±25.23 sec, respectively; not significantly different), while diabetic groups (DM and DM+HDACi) showed significant decreased fall latency compared to nondiabetic group (NDM versus DM: 281.69±13.27 sec versus 256.61±21.99 sec; NDM versus DM+HDACi: 281.69±13.27 sec versus 254.77±25.23 sec; P<0.01 for both) (FIG. 2C). These data indicate that direct injection of HDAC inhibitor improves mechanical allodynia and prevents thermal hypoalgesia in mice with diabetic neuropathy but has no recovery effect on rotarod performance.

### Administration of Belinostat Increases Vascularization in Diabetic Nerves.

To examine changes in the functional blood vessels at the histological level, sciatic nerves were harvested 18 weeks after treatment, following systemic injection of FITC-conjugated BSL1 to visualize functional blood vessels. Whole mount longitudinal images demonstrated that the nerves from nondiabetic mice (NDM) were well vascularized and had clear epineurial longitudinal networks and penetrating branches running from epineurial to endoneurial vessels. However, in diabetic mice (DM), both the epineurial and endoneurial vessels, and especially small branches from the epineurial vessels, were markedly decreased leaving focal areas very poorly vascularized. In contrast, belinostat injected diabetic mice (DM+HDACi) showed increased vascularity in sciatic nerves, particularly small branches of epineurial blood vessels.

### Protective Effect of Belinostat on Diabetes-Induced Intraepidermal Nerve Fiber Loss.

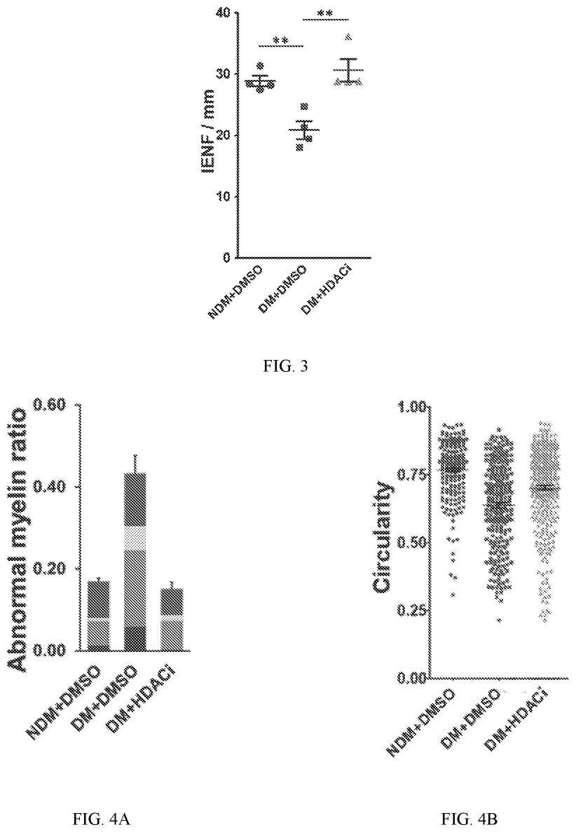

Diabetic mice exhibited robust reduction of intraepidermal nerve fibers (IENF), which is consistent with findings from skin biopsy sample from diabetic patients showing that reduction of blood vessels is closely associated with axonal degeneration. To evaluate the protective effect of belinostat on diabetic intraepidermal nerve fiber, we stained IENF in hind paw plantar skin with pan-neuronal marker PGP9.5 and counted the number of fibers throughout the epidermis relative to length of tissue. The DM group mean showed 27.7% reduced number of IENF compared to NDM group (NDM versus DM: 28.85±0.85 IENF/mm versus 20.87±1.44 IENF/mm; P<0.01), while no differences in IENF levels were observed between NDM and DM+HDACi group (NDM versus DM+HDACi: 28.85±0.85 IENF/mm versus 30.61±1.83 IENF/mm; not significantly different from NDM) (FIG. 3). These data demonstrate that administration of belinostat protects against diabetes-induced IENF loss.

### Belinostat Treatment Ameliorates Ultrastructural Morphology of Myelinated Fibers Altered in Diabetic Nerves.

It was examined whether belinostat could affect the ultrastructure of diabetic nerves, which is characterized by morphological alterations in the myelin sheath, a hallmark of DN. Accordingly, light microscopy and transmission electron microscopy were performed on sciatic nerves obtained at 18 weeks after STZ injection. The alternation of myelination pattern was categorized into four groups: decompaction, infolding, focal lysis, and splitting. Ultrastructure of the sciatic nerve in the NDM group showed low percentage of alternated myelination pattern (decompaction: 8.97%, infolding: 0.86%, focal lysis: 5.84%, splitting: 1.29%; n=236) (FIG. 4A). In the DM group, the rate of alternation in myelinated axons was significantly increased (decompaction: 12.90%, infolding: 5.93%, focal lysis: 18.64%, splitting: 5.87%; n=429) (FIG. 4A). However, the diabetic animals treated with belinostat group (DM+HDACi) showed markedly fewer morphologic alterations in ultrastructural features of myelin sheath and axons (decompaction: 6.35%, infolding: 1.49%, focal lysis: 7.22%, splitting: 0%; n=406) (FIG. 4A).

Axonal atrophy is associated with slowing of nerve conduction but is difficult to distinguish from loss of large diameter axons. A reduced index of circularity and an increased g-ratio supports the axonal atrophy. The index of circularity in diabetic groups (both DM and DM+HDACi) showed 20% and 10.3% reduced axonal circularity compared to nondiabetic group (NDM), respectively (NDM versus DM: 0.78 versus 0.63, P<0.001; NDM versus DM+HDACi: 0.78 versus 0.70, P<0.001), while DM+HDACi group showed higher circularity than DM group (DM versus DM+HDACi: 0.63 versus 0.70; P<0.001) (FIG. 4B). However, g-ratio of myelinated sciatic nerve were similar among experimental groups (NDM versus DM versus DM+HDACi: 0.64±0.0093 versus 0.63±0.014 versus 0.65±0.0044; not significantly different).

These data indicate that the axonal atrophy is not a prominent feature in STZ-induced diabetic mice. A loss of large diameter axons in the peripheral nerve system is the most frequent consequence of metabolic diseases such as diabetes. Analysis of myelinated axons of the sciatic nerve showed a significant decrease in axon diameter of DM group compared to NDM group suggesting a loss of large diameter axons in diabetes (NDM versus DM: 4.49±0.14 m versus 3.56±0.10 m; P<0.001). Belinostat treatment significantly prevented the loss of large diameter axons (DM versus DM+HDACi: 3.56±0.10 m versus 4.17±0.10 m; P<0.001), and even DM+HDACi group showed a similar diameter of nondiabetic group (NDM versus DM+HDACi: 4.49±0.14 m versus 4.17±0.10 m; not significantly different). Taken together, these finding suggest that HDAC inhibitors such as belinostat ameliorates the ultrastructure of the myelin sheath and axons in diabetic sciatic nerves.

1. A method of treating or preventing diabetic neuropathy comprising administering an effective amount of an epigenetic modifying agent to a subject in need thereof.

2. The method of claim 1 wherein the epigenetic modifying agent is a histone deacetylase inhibitor.

3. The method of claim 1 wherein the agent is belinostat.

4. The method of claim 1 wherein the agent is quisinostat.

5. The method of claim 1 wherein the agent is vorinostat.

6. The method of claim 1, wherein the subject is at risk of, exhibiting symptoms of, or diagnosed with diabetic neuropathy, diabetes, or prediabetes.

7. The method of claim 1, wherein the agent is administered in the absence of autologous stem cells or the agent is administered in combination with autologous stem cells.

8. The method of claim 1, wherein the subject is a human subject.

9. A method of reducing pain comprising administering an effective amount of an epigenetic modifying agent to a subject in need thereof.

10. The method of claim 9 wherein the epigenetic modifying agent is a histone deacetylase inhibitor.

11. The method of claim 9 wherein the agent is belinostat.

12. The method of claim 9 wherein the agent is quisinostat.

13. The method of claim 9 wherein the agent is vorinostat.

14. The method of claim 9, wherein the subject is at risk of, exhibiting symptoms of, or diagnosed with diabetic neuropathy, diabetes, or prediabetes.

15. The method of claim 9, wherein the agent is administered in the absence of autologous stem cells or the agent is administered in combination with autologous stem cells.

16. The method of claim 9, wherein the subject is a human subject.
